(19) United States
(12) Patent Application Publication
(10) Pub. No.: US 2004/0126405 A1
(43) Pub. Date: Jul. 1, 2004

(54) Engineered scaffolds for promoting growth of cells

(75) Inventors: Ronald A. Sahatjian, Lexington, MA (US); Michael S. Banik, Bolton, MA (US); Sheng-Ping Zhong, Northborough, MA (US); Toby Freyman, Watertown, MA (US); Liem Vu, Needham, MA (US); Kinh-Luan D. Dao, Brockton, MA (US); Yem Chin, Burlington, MA (US)

Correspondence Address: HOFFMANN & BARON, LLP, 6900 JERICHO TURNPIKE, SYOSSET, NY, 11791, US

(73) Assignee: SCIMED Life Systems, Inc.

(21) Appl. No.: 10/334,096

(22) Filed: Dec. 30, 2002

(51) Int. Cl. C12N 5/00; C12N 5/02

(52) U.S. Cl. 424/423; 435/393

(57) ABSTRACT

A three dimensional cell scaffold is provided including a biocompatible polymer formed from a plurality of fibers configured so as to form a non-woven three dimensional open celled matrix having a predetermined shape, a predetermined pore volume fraction, a predetermined pore shape, and a predetermined pore size, with the matrix having a plurality of connections between the fibers.

### FIELD OF THE INVENTION

The present invention relates generally to tissue engineering, specifically to three-dimensional scaffolding for cell and tissue culture. In particular, the present invention relates to a non-woven polymeric spun scaffold for use in cell transplantation and/or organ reconstruction.

### BACKGROUND OF THE INVENTION

A wide variety of medical conditions exist that can be improved or corrected by the use of three dimensional tissue scaffolding that serves as a support system for cells intended to grow and replace missing and/or damaged tissue. The medical conditions can vary from acute trauma caused by car accidents to degenerative disease in which tissue structure and function are compromised or lost. The challenge has been to identify and develop systems that will replace or enable the body to regenerate lost or damaged tissue.

A three dimensional scaffold desirably possesses sufficient mechanical strength to maintain its form when exposed to forces such as those exerted by cells in its interior as well as pressure from surrounding tissue when implanted in situ. The scaffold is non-toxic, biocompatible and serves as a suitable substrate to allow seeded cells to attach and proliferate uniformly throughout the structure. The cells are then able to differentiate and perform the function of the native cells they are intended to replace or supplement. Native cells integrate into the scaffold, any necessary vasculature develops, and ultimately the cell scaffold performs the function(s) of the tissue it was designed to replace or supplement. Desirably, the scaffold gradually dissolves as new cellular growth occurs, leaving functional replacement tissue in its place.

Early efforts to regenerate cells to form viable organs and/or organ parts were focused on providing appropriate cells in a biocompatible suspension. For example, chondrocyte cell suspensions were mixed with dry alginate powder to form a gel which when injected into experimental animals, showed evidence of cartilage formation without migration of the material to sites remote from the point of injection. Atala et al., Journal of Urology, 150:745-747 (August 1993). A limitation to this type of procedure is that an injected gel is expected to form a random shape which may or may not be useful in the tissue to be regenerated.

Further developments in the art have included forming scaffolding for cells which has a predetermined three dimensional structure. Scaffold morphology is directly related to the method and materials used to fabricate the structure. Three-dimensional scaffolds are known to be formed from natural or artificial polymers or combinations thereof, or from what is known as inorganic composites. A variety of techniques are currently available for making tissue scaffolding and include fiber bonding, solvent casting and particulate leaching, membrane lamination, melt molding, polymeric/ceramic fiber composite foams, phase separation, and in situ polymerization. R. C. Thompson, “Polymer Scaffold Processing,” in Principles of Tissue Engineering, Eds. R. Lanza et al., R. G. Landis Co. (1997). Depending on the raw materials and methods used, scaffolding can be made in a variety of shapes and sizes.

In order for a scaffold to perform properly, it must possess certain morphological and other characteristics. Among the most significant morphological characteristics of open celled materials are relative density and the correlative pore volume fraction, cell shape and uniformity, and to a lesser extent, cell size. Cells or pores are the void spaces within the material. Open celled materials mean the cells connect through open faces. In contrast, closed cell materials are made of cells that are closed off from one another. Relative density ρ*/ρₛ is the density of the cellular material, ρ*, divided by that of the solid from which the cell walls are made ρₛ. Pore volume fraction is that portion of the material occupied by the pore space or 1−ρ*/ρₛ. As relative density increases, the cell walls thicken, the pore space shrinks, and pore volume fraction is reduced. Typical open celled materials possess a relative density of about 0.3 or less.

In designing a material for use as a cellular scaffold, it is important for the pores to be of a sufficiently large size so as to allow cells (i.e., living cells) to maintain their shape within the structure. Additionally, an open cell configuration and a large pore volume fraction are desirable in order to allow a cell suspension to fully penetrate the structure and thus permit cell seeding and/or cell migration throughout the material. An insufficient pore size and/or pore volume fraction will restrict cells from gaining uniform access throughout the scaffold structure. Furthermore, free access of nutrients to the cells as well as efficient removal of waste products formed as a result of cellular metabolism will be impeded. Related to pore volume fraction and porosity is the surface area to volume ratio within the structure. It is believed that a high surface area to volume ratio encourages adhesion of cells to the scaffold surfaces.

It is also important for the pores to be relatively uniform in size. This assures the pores are large enough to accommodate the living cells uniformly throughout the scaffold. Furthermore, a lack in uniformity in cell shape and size, referred to as shape anisotropy, results in an anisotropic scaffold with irregularities in its properties. These irregularities may be undesirable in certain applications. For example, elongated cells, having greater cell diameter in a particular direction, can cause the resultant scaffold to be twice as stiff in the elongated as opposed to the other direction. Gibson and Ashby, Cellular Solids—Structure and Properties, 2nd ed., Cambridge Univ. Press (1997). Thus, an anisotropic scaffold may be undesirable when it is important to maintain a uniform stiffness in the scaffold. Furthermore, if the anisotropy results in pores too small to accommodate cells, then there will be non-uniform and potentially insufficient cell proliferation throughout the scaffold. Limitations of currently available tissue scaffolds include the inability to provide scaffolds having an optimal pore volume fraction, uniformity of cell shape and size, and a sufficient surface area to volume ratio.

In addition to sufficient morphology, in order for a tissue scaffold to be useful, it must be relatively non-toxic or biocompatible. As used herein a material is biocompatible if it does not significantly compromise the function of the host organism. This is especially important both when initially seeding the scaffold and during degradation of the scaffold when toxic breakdown products (such as acids) are often generated. If residual solvents remain in the scaffold after initial manufacture, then it may be difficult to successfully seed the scaffold with cells. Furthermore, when the scaffold degrades, it is important that the material either degrade at a rate sufficiently slow to avoid toxic buildup of breakdown produces, or have degradation products which are non-toxic to cells.

One known scaffold is made using phase separation upon freeze-drying. In this method, the base material is dissolved in a suitable solvent and rapidly frozen. The solvent is removed by freeze-drying leaving behind a porous structure. One type of scaffold made in this way is a porous collagen sponge having pores between about 50 and about 150 μm. Pieper et al., Biomaterials, 20:847-858 (1999). A disadvantage of this scaffold is that the shape, size and interconnectedness of the pores is randomized due to the freeze drying process. As a result, dead end channels and/or pores that are too narrow can be formed in which cells are either trapped without access to nutrients or unable to uniformly populate the scaffold. This non-uniform structure is not optimal for uniform distribution of cells throughout the scaffold.

Known synthetic polymer scaffolds may also be manufactured by freeze-drying and include polylactic acid foams with porosity of up to about 95% having an anisotropic tubular morphology and an internal ladder-like structure containing channels ranging from several tens of microns to several hundred microns in diameter Zhang et al., J. Biomed. Mater. Res., 45:285-293 (1999). Polyglycolic acid foams having a porosity of 90%-95%, average pore sizes ranging from about 15 microns to about 35 microns, and pores of up to about 200 microns are also known. Whang et al., Polymer, 36:837-842, (1995).

These synthetic polymer scaffolds suffer the same disadvantages as their natural polymer counterparts. Namely, although these scaffolds are relatively porous, the material resists uniform distribution of seeded cells throughout the entire structure. In addition, foams formed in this way often lack the necessary mechanical strength to serve as scaffolds to replace hard tissue such as bone.

Certain biologically active agents are useful in improving the performance of three dimensional scaffolds. For example, extracellular matrix (ECM) molecules consisting of secreted proteins and polysaccharides occupy the intercellular space and bind cells and tissues together. Cells can attach to matrix proteins by interacting with them through cell adhesion molecules such as integrins. It is believed that the presence of ECM molecules in a three dimensional scaffold may act to improve cell adhesion. In addition, the presence of signaling and ECM molecules can encourage cells to perform their differentiated tissue specific functions. These properties can facilitate the scaffold to serve its function as either a living tissue equivalent or as a model tissue system.

Scaffolds are often seeded with cells prior to implantation into a mammal. One function of the seeded cells and their associated protein products is to direct migration of indigenous or native cells from neighboring tissue onto the scaffold and ultimately to replace the scaffold with native cells and tissue. It is also possible to seed cells onto the scaffold and later kill the seeded cells by freezing or freeze drying the scaffold construct prior to implantation. In this way, living material is eliminated from the scaffold, but the deposited proteins, such as ECM molecules, are left behind in their natural states.

U.S. Pat. No. 6,179,872 B1 to Bell et al., discloses a biopolymer matt formed from biocompatible and biodegradable bipolymers formed as a densely packed random array of fibrils or bundles of fibrils. The fibrils are made by orderly side-by-side associations of the polymer molecules. The matt is made by applying a liquefied form of the biopolymer over a mesh stainless steel screen, drying the biopolymer, and removing the matt from the screen after it has solidified. The matt may be seeded with tissue specific cells and bioactive agents such as ECM proteins before being introduced into a recipient. This material is primarily a two dimensional structure and has limited application in replacing thick tissues.

U.S. Pat. No. 6,333,029 to Vyakarnam et al. discloses a three-dimensional porous foam for use in tissue engineering having a gradient architecture through one or more directions. The gradient is created by blending polymers to create a compositional gradient by timing onset of a sublimation step in the freeze drying process used to form the foam. One or more growth factors may be incorporated into the structure. However, this material suffers from the same disadvantages of the other prior art foams, including the possibility of toxic solvents remaining in the foam and a lack of sufficiently interconnected channels.

Although a variety of tissue scaffolding is presently available, there remains a critical need for a tissue scaffold with optimal performance in satisfactorily replacing damaged or lost tissue including a biocompatible structure that retains adequate mechanical strength while providing sufficient pore volume fraction, pore size, pore shape, surface area to volume ratio, and uniformity of internal architecture necessary for cellular infiltration.

### BRIEF DESCRIPTION OF THE DRAWINGS

FIG. 1 is a top perspective view of an apparatus for making a three dimensional non-woven polymeric scaffold according to the invention.

FIGS. 2A to 2D are exploded top views of embodiments of internal architecture of the non-woven polymeric scaffold according to the invention.

### SUMMARY OF THE INVENTION

The present invention provides a three dimensional cell scaffold including a biocompatible polymer formed from a plurality of fibers configured so as to form a non-woven three dimensional open celled matrix having a predetermined shape, a predetermined pore volume fraction, a predetermined pore size and a predetermined pore shape, with the matrix having a plurality of connections between the fibers.

In a still further aspect of the present invention, a method for regenerating tissue in a mammal is provided including implanting the cell scaffold of the present invention into the mammal.

Additionally, a method of treating Gastro Esophageal Reflux Disease (GERD) is provided including forming a biocompatible polymeric matrix formed from a plurality of fibers configured so as to form a non-woven three dimensional open celled tubular matrix. The matrix has a predetermined pore volume fraction, a predetermined pore shape, a predetermined pore size sufficient to accommodate a diameter of esophageal epithelial cells, and a plurality of connections between the fibers. The matrix is seeded with esophageal epithelial or stem cells and implanted into a mammalian esophageal space.

In another aspect of the invention, a method of removing diseased esophageal tissue is provided including the steps of: (a) forming a biocompatible polymeric matrix formed from a plurality of fibers configured so as to form a non-woven three dimensional open celled tubular matrix, with the matrix having a predetermined pore volume fraction, a predetermined pore shape, a predetermined pore size, and including a plurality of connections between the fibers, (b) treating the matrix with a predetermined concentration of a cell destroying compound; and (c) implanting the matrix into a mammalian esophageal space.

In a further aspect, a three dimensional cell scaffold of the invention is formed from the steps of: (a) admixing at least a biocompatible polymer with a compatible solvent to form a flowable polymer mixture; (b) applying at least one fiber formed from the polymer mixture to an application table capable of motion in at least a first plane (x) and a second plane (y) perpendicular to the first plane; and (c) controlling movement of at least the table so as to form a three dimensional non-woven matrix of fibers having a predetermined pore size, a predetermined pore shape, a predetermined pore volume fraction, and a plurality of connections between the fibers.

In addition, a tissue modeling kit is provided including a cell scaffold according to the invention and a plurality of viable cells from a tissue to be modeled, wherein the viable cells are cultured in the cell scaffold.

In a still further aspect of the present invention, a method of testing toxicity to a tissue is provided including the steps of: (a) forming a cell scaffold according of the invention, wherein a shape of the scaffold resembles at least a portion of a tissue to be tested; (b) culturing cells derived from the tissue in the cell scaffold; (c) administering a predetermined dosage of a test agent to the cell scaffold; and (d) measuring a cellular response to the dosage.

With the foregoing and additional features in mind, this invention will now be described in more detail, and other benefits and advantages thereof will be apparent from the following detailed description when taken in conjunction with the accompanying drawings in which like numerals represent like elements throughout the several views.

### DETAILED DESCRIPTION OF THE PREFERRED EMBODIMENTS

In order to optimize successful cell attachment, growth, and differentiation, a tissue scaffold desirably possesses suitable internal architecture including pore shape and size, pore volume fraction, and surface area to volume ratio. The scaffold is biocompatible so as to avoid eliciting a significant detrimental effect in the host, and additionally, it desirably degrades in a rate and a fashion so as to avoid causing cell death from toxic degradation products.

The present invention features a tissue scaffold formed from biocompatible natural polymers, synthetic polymers, or combinations thereof, into a non-woven open celled matrix having a substantially open architecture, which provides sufficient space for cell infiltration while maintaining sufficient mechanical strength to withstand the contractile forces exerted by cells growing within the scaffold during integration of the scaffold into a target site within a host.

It is contemplated as within the invention to use the polymers alone, as copolymers, or blends thereof. The polymers may be biodegradable or biostable or combinations thereof. As used herein, “biodegradable” materials are those which contain bonds that may be cleaved under physiological conditions, including enzymatic or hydrolytic scission of the chemical bonds.

Suitable natural polymers include polysaccharides such as alginate, cellulose, dextran, pullane, polyhyaluronic acid, chitin, poly(3-hydroxyalkanoate), poly(3-hydroxyoctanoate) and poly(3-hydroxyfatty acid). Also contemplated within the invention are chemical derivatives of said natural polymers including substitutions and/or additions of chemical groups such as alkyl, alkylene, hydroxylations, oxidations, as well as other modifications familiar to those skilled in the art. The natural polymers may also be selected from proteins such as collagen, zein, casein, gelatin, gluten and serum albumen.

Suitable synthetic polymers include polyphosphazenes, poly(vinyl alcohols), polyamides, polyester amides, poly(amino acids), polyanhydrides, polycarbonates, polyacrylates, polyalkylenes, polyalkylene glycols, polyalkylene oxides, polyalkylene terephthalates, polyortho esters, polyvinyl ethers, polyvinyl esters, polyvinyl halides, polyesters, polylactides, polyglyxolides, polysiloxanes, polycaprolactones, polyhydroxybutrates, polyurethanes, styrene isobutyl styrene block polymer (SIBS), and copolymers and combinations thereof.

Biodegradable synthetic polymers are preferred and include poly α-hydroxy acids such as poly L-lactic acid (PLA), polyglycolic acid (PGA) and copolymers thereof (i.e., poly D,L-lactic co-glycolic acid (PLGA)), and hyaluronic acid. Poly α-hydroxy acids are approved by the FDA for human clinical use. It should be noted that certain polymers, including the polysaccharides and hyaluronic acid, are water soluble. When using water soluble polymers it is important to render these polymers partially water insoluble by chemical modification, for example, by use of a cross linker.

The polyanhydrides and polyesters, such as PLA and PGA, contain labile bonds and are known for their hydrolytic reactivity. The hydrolytic degradation rates of these polymers can generally be regulated by changing the polymer backbone and sequence structure accordingly.

Further examples of suitable biodegradable elastomers are described in U.S. Pat. Nos. 4,045,418, 4,057,537 and 5,468,253, which are hereby incorporated by reference in their entireties. In addition, non-limiting examples of some useful composites of natural and synthetic polymeric materials for scaffolding applications are disclosed in Chen, G. et al., Advanced Materials, 12:455-457, (2000), which is hereby incorporated by reference.

The scaffolding of the present invention is made by extruding a biocompatible polymer dissolved in a suitable solvent or melted to form a viscous solution from which a continuous fiber may be drawn. The solution is extruded under pressure and fed at a certain rate through an opening or openings in a dispenser of a predetermined size to form a fiber or fibers. A desired fiber thickness, typically from about <1 to about 100 microns, preferably from about 3 to about 30 microns, is formed and drawn by the actions of a moveable table having three degrees of freedom of movement that is controlled by using computer assisted design (CAD) software. The table is capable of motion in two or three planes, and is referred to herein as the application table or simply as the table. The rate of elongation and stretch of the fiber, if any, is similarly regulated by the programmed motion of the table in relation to the spinneret. The method is more fully disclosed in the U.S. patent application entitled “Porous Melt Spun Polymeric Structures and Methods of Manufacture,” Ser. No. ______ filed ______ under attorney docket no. 498-277, the entirety of which is hereby incorporated by reference.

The apparatus and method of the present invent ion is capable of forming a porous matrix which is similar in size, shape, and strength to that formed by the method of the prior art. However, the apparatus and method of the present invention has capabilities well beyond that of the prior art methods. Whereas the prior art method forms one particular internal architecture, which is often random and uncontrolled, the present invention is not so limited. Specifically, the method of the present invention allows for a wide variety of specific predetermined internal architectures. The method allows for specific design of pore channel configurations such as channel shape, size, and channel inter-connections. Each of these parameters may be predetermined by selecting appropriate movements of the moveable table.

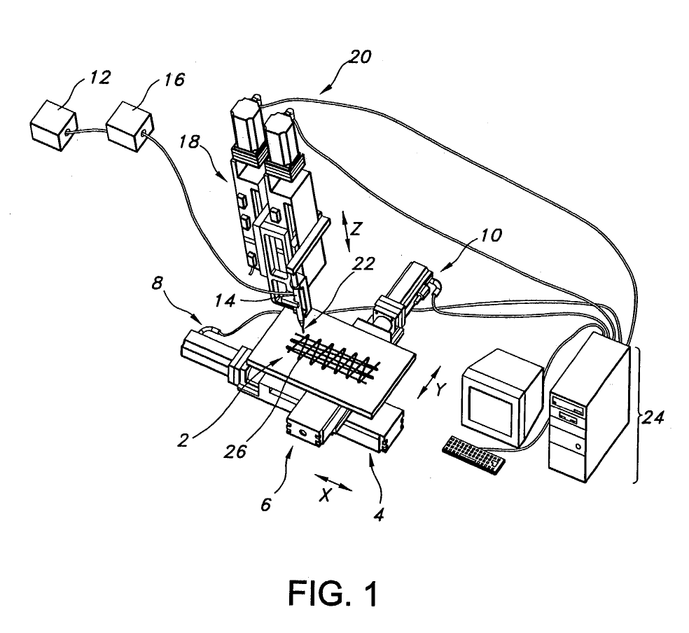

Referring now to FIG. 1, a perspective view of the apparatus for making the porous matrix of the present invention is shown. The moveable table 2 is operatively attached to an x drive member 4 and a y drive member 6. Movement of the drive members 4, 6 is achieved by an x control member 8 and a y control member 10. A holding chamber 12 houses the polymer which is fed into an applicator 14 via a pump 16. The liquid polymer is fed through the applicator 14 onto the table 2. The applicator 14 may remain stationary, or may be moved in relation to the table via a z drive member 18 which is controlled by a z control member 20. Movement of the table 2 results in deposition of a fiber or fibers 22 in a layer 26 on the table 2.

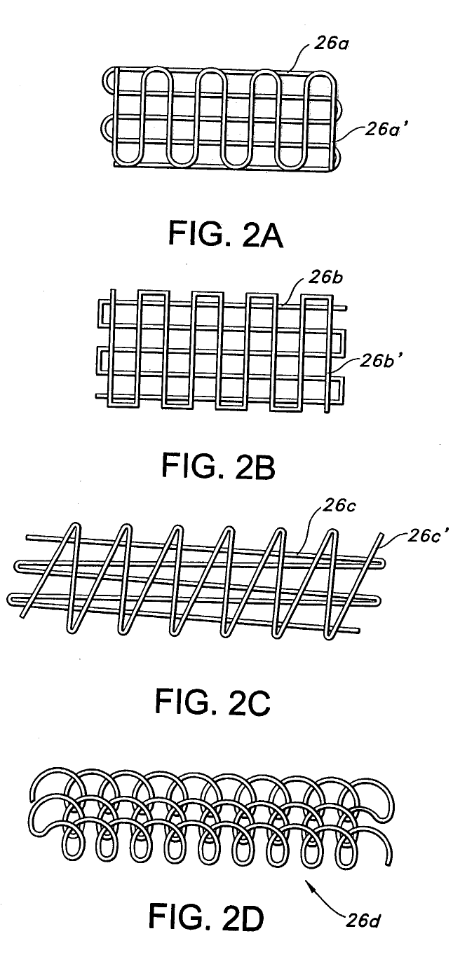

In operation, the table moves in a predetermined pattern so as to produce a particular predetermined fiber design and pore volume fraction. A three dimensional structure can be built up by repeating the motion of the table as many times as required to achieve the desired shape, size, and thickness of the matrix. Referring now to FIGS. 2A-2D, representative designs of internal architectures of the scaffold of the invention are shown. In FIG. 2A, a first sine wave pattern is shown in a first layer 26a with a second sine wave pattern in a second layer 26a′, which is placed at a 90° angle with respect to the first layer 26a. In FIG. 2B, a step wave pattern in layers 26b and 26b′ is shown. In FIG. 2C, a saw toothed wave pattern in layers 26c and 26c′ is shown. FIG. 2D shows a concentric loop layer 26d. The patterns may be used alone or in appropriate combination, depending on the intended use of the scaffold. Some Examples of designs of scaffolds for particular applications are discussed in further detail below.

It is also possible to form the scaffold by a spinning technique such as that described in U.S. Pat. No. 4,475,972 to Wong and U.S. Pat. No. 5,755,774 to Pinchuk, the entireties of which are herein incorporated by reference. This method is particularly useful for tubular forms. Briefly, polymer in solution is extruded into fibers from a dispenser known as a spinneret onto a rotating mandrel. The spinneret system is reciprocated along the longitudinal axis of the mandrel at a controlled pitch angle, resulting in a non-woven structure where each fiber layer is bound to the underlying layer. The fibers can be spun in layers onto the mandrel to a desired thickness. The internal diameter can be adjusted, for example, by adjusting the diameter of the mandrel.

The scaffold of the present invention can be produced from fibers formed by diluting the desired polymer in an appropriate solvent. Optionally, a cross-linking agent may be added from a separate source to the solution just prior to application of the mixture to the table so as to assist in fiber formation. In particular, water soluble polymers including polysaccharides such as alginate, require a cross-linker. Suitable cross-linking agents for these polymers include metal ion solutions, such as the salts of calcium, copper, aluminum, magnesium, strontium, barium, tin, and zinc. Particularly desirable cross linking agents for natural polymers, particularly alginate include calcium chloride (CaCl₂), strontium chloride (SrCl₂) and calcium gluconate (Ca-Gl). Cross linking agents suitable for use with collagen include aldehydes such as gluteraldehyde and carbodiimides. When using a cross-linker, it is important to introduce the cross-linker just prior to or just after formation of the fiber. For example, it is possible to have a two chamber feed design in which the polymer solution and cross-linking agent are introduced just prior to entry into the spinneret. Alternatively, it may be possible to form a fiber from the uncrosslinked material and then pass the fiber into a bath containing the cross-linker prior to application on the table.

It is contemplated as within the invention to use the polymers alone, as copolymers, or blends thereof. Selection of the polymer combinations will depend upon the particular application and include consideration of such factors as desired tensile strength, elasticity, elongation, modulus, toughness, viscosity of the liquid polymer, whether biodegradable or permanent structures are intended, and the like to provide desired characteristics.

One having skill in the art may select appropriate combinations based on the desired characteristics of the matrix and what is known in the art regarding the individual polymers of interest. For example, polyanhydrides and polyvinyl chlorides are known to introduce flexibility into a polymer. It is possible, therefore, to use a small amount of certain polymers as additives to impart desired properties to the main polymer or polymer blend. For example, by adding some polyanhydride to a PLA polymer, flexibility of the structure formed thereof is increased. Small amounts of a non-biodegradable polymer may be added to a biodegradable polymer without compromising the biodegradability of the final material formed thereof. Selection of polymer blends, copolymers, and additives will be based on the particular end use of the polymeric matrix structure and can be made accordingly by one having ordinary skill in the art. It is therefore within the contemplation of the invention to employ multiple polymers, polymer blends, copolymers, and additives to maximize desirable matrix properties. In one desirable aspect of the invention, a matrix is made from a polymer including about 70% polylactic acid and about 30% polyurethane.

Furthermore, it is specifically contemplated by the inventors that matrices of the present invention may be created by alternately applying or simultaneously applying more than one polymer or copolymer. For example, it is possible to apply two different polymeric fibers by using two applicators to apply two different polymers or polymer blends simultaneously. Alternatively, it is possible to apply a first polymeric fiber in a first layer or layers, and apply a subsequent second polymeric fiber or fibers in a subsequent layer or layers. By alternating the polymer, a matrix can be made having varying properties depending on the distribution of each of the polymer, copolymer or blends within the matrix.

Varying the size of the openings of the applicator, rate of feed of the liquid, and movement of the table, allows for a three dimensional scaffold to be formed having any desired shape and size. Generally, scaffolds made in accordance with the present invention have a thickness of about 0.1 to about 10 mm and more desirably up to a thickness of about 30 mm. Moreover, the pore size, pore shape, and pore volume fraction may similarly be controlled by the rate of feed, size of openings, and movements of the table, and can be varied in a predetermined fashion to fit a particular application. For example, the scaffolding of the present invention may be formed as a sheet having a uniform pore volume fraction, pore shape, and pore size throughout the sheet. Alternatively, the scaffolding may be formed as a tube having a gradient beginning with a first predetermined pore volume fraction and pore size at an internal diameter of the tube which gradually changes along its cross section to a second predetermined pore volume fraction and pore size at an external diameter of the tube. The pore shape may be uniform throughout or progressive along a dimension of the scaffold. It is also possible to program the movements of the spinneret and table to provide a scaffolding having a randomized structure within any predetermined ranges of pore shapes, pore sizes and pore volume fraction.

Any material which is biocompatible, may be formed into fibers, and degrades at a suitable rate may be used. The pore volume fraction is selected so as to encourage cellular penetration and growth throughout the scaffold. Generally a PVF of from 60>98% is desirable. Particularly advantageous is a PVF of greater than 80%. The pore volume fraction may be uniform or non-uniform. It may, for example, be desirable to limit access of cells to a portion of a scaffold. In this instance, a scaffold may be designed having a portion with a pore volume fraction which prevents coinflux of cells to that portion.

The pore volume fraction (PVF) is selected so as to encourage cellular penetration and growth throughout the scaffold. Generally, a PVF of from about 60 to 98% is desirable. Particularly advantageous is a PVF greater than about 80%. The pore volume fraction may be uniform or non-uniform. It may, for example, be desirable to limit access of cells to a portion of a scaffold. In this instance, a scaffold may be designed having a portion with a pore volume fraction which prevents influx of cells to that portion.

The scaffold of the present invention may be made uniformly of a single polymer, co-polymer or blend thereof. However, it is also possible to form a scaffold according to the invention of a plurality of different polymers. There are no particular limitations to the number or arrangement of polymers used in forming the scaffold. Any combination which is biocompatible, may be formed into fibers, and degrades at a suitable rate, may be used. It is possible, for example, to apply polymers sequentially. In this case, a first polymer is dispensed on the table to form a pre-determined first pattern followed by a second polymer dispensed on the table to form the same or a different second pattern. In a desirable aspect of the invention a first biodegradable polymer can be formed into a partial scaffold design followed by a second more biostable polymer to form the complete scaffold. Particularly desirable is to form a scaffold having a biostable polymer portion of the scaffold sandwiched inside two biodegradable polymer portions.

Desirably, the biodegradable polymer portion is one of collagen, PLA, or PGA, and the biostable portion is a SIBS block polymer. An advantage of using a biostable polymer in combination with a biodegradable polymer is that the biodegradable polymer can degrade over time allowing for full integration of cellular material in its place. The remaining biostable polymer portion may then remain and serve a support function to the newly integrated cellular material. Thus, this aspect of the invention is particularly beneficial for use with any organ in which mechanical strength of the tissue is important.

It is also possible to use a combination material of a polymeric material and a non-polymeric material in forming the scaffold. For example, when replacing bone or cartilage containing material, it is important for the scaffold to possess mechanical strength. Certain ceramic powders are known to be useful in providing mechanical strength to prostheses. To this end, in one aspect of the invention, a ceramic powder is formed into a solution in combination with a polymeric binder such as polyacrylate or PMMA. The polymeric part of the mixture will allow for the solution to be formed into fibers for application onto the table to form the pores of the scaffold. Interspersed within this polymeric matrix can be support structures made from the ceramic solution. The polymeric material is desirably biodegradable. In use, cells will enter and proliferate the biodegradable portion of the scaffold and ultimately be replaced therewith. However, the support structure within the scaffold will remain. It is also possible to use the combination material in further combination with other polymers as described previously.

In one aspect of the invention, the scaffold of the present invention may be used in conjunction with one or more support members which assist in providing support of the scaffold. Support members include, but are not limited to, stents, posts, hooks, bands and coils. These may be permanent or temporary structures as long as they are biocompatible. The open celled polymeric scaffold matrix of the present invention may be formed around the support member. Alternatively, the matrix may be formed, seeded with cells, and the support member can be added to the scaffolding prior to implantation into a recipient in need thereof.

The spun polymer scaffold can be seeded with cells prior to use. One having skill in the art will appreciate how to seed cells into the scaffold. For example, static cell seeding may be used wherein cells are delivered to the scaffold by first suspending them in tissue culture medium. This suspension is then applied onto one or more of the surfaces of the scaffold and allowed to enter the pores of the scaffold. Alternatively, dynamic cell seeding may be used in which the scaffold is placed in a vessel containing a cell suspension. The vessel is shaken so as to distribute the cell suspension evenly throughout the scaffold.

The polymer scaffolds may be seeded with mammalian cells. However, it is contemplated that the scaffold may be seeded with any of a variety of cells. The term cell as used herein means any preparation of living tissue, inclusive of primary tissue explants and preparations thereof, isolated cells, cell lines (including transformed cells) and host cells. Preferably, autologous cells are employed. However, xenogeneic, allogenic, syngeneic cells, or stem cells may also be useful.

In one aspect of the invention, the scaffold is used in vivo as a prosthesis or implant to replace damaged or diseased tissue. The scaffold may be formed into an appropriate shape and then introduced or grafted into recipients such as a mammalian and in particular a human recipient. The structure of the scaffold can be designed to mimic internal as well as external configurations. Further modifications to the design may be made after the polymer is formed, including cutting the matrix to the proper size. Any of a variety of tools may be used in this regarding including scissors, a scalpel, a laser beam, and the like. Non-limiting examples of such shapes include sheets, tubes, cylinders, spheres, semi-circles, cubes, rectangles, wedges, and irregular shapes. Once the introduced scaffold is occupied by cells, e.g., host cells, it serves as functional tissue. When used in vivo, it is preferable that the scaffold biodegrade after sufficient host tissue has been formed.

It is further desirable to pre-seed the scaffold prosthesis prior to introduction into the recipient. This is helpful in speeding integration of the scaffold, recovery of repair tissue, and replacement of the damaged or missing tissue. Furthermore, in embodiments where the cells are not autologous, it may be desirable to administer an immunosuppressant drug in order to minimize risk of rejection. Such agents may be included within the seeding composition.

In a preferred aspect of the invention, normal or non-disease state autologous host cells are harvested from the intended recipient and processed under sterile conditions for later use in seeding the scaffold. Methods for seeding the scaffold are known in the art. Preferably, the cell seeded scaffold is placed in a bioreactor to allow the cells to proliferate prior to the scaffold being implanted into a patient. The method of Caplan, as disclosed in U.S. Pat. No. 5,486,359, is instructive. Cells grown in the scaffold of the invention have morphologies characteristic of cells of three dimensional tissues and can form normal intercellular relationships, i.e., intercellular relationships like those in the tissue from which they are derived or obtained. It is also possible to encapsulate the cells with a protective polymer coating before introduction into the scaffold.

Non-limiting examples of tissues which can be repaired and/or reconstructed using the scaffolding described herein include nervous tissue, skin, vascular tissue, cardiac tissue, pericardial tissue, muscle tissue, ocular tissue, periodontal tissue, connective tissue such as bone, cartilage (articular, meniscal, septal, tracheal), tendon, and ligament, organ tissue such as breast, pancreas, stomach, esophageal, vascular, kidney, ocular and hepatic, glandular tissue such as pancreatic, mammary, and adrenal, urological tissue such as bladder and ureter, and digestive tissue such as intestinal.

The scaffold may be used as a substrate for the growth of cells appropriate for the particular application. For example, scaffolding may be seeded with osteoblasts to repair bone defects, mesothelial cells to repair a pericardial membrane, mesothelial cells to repair the abdomen, epithelial cells to repair skin, epithelial cells to repair esophagus, and so on. Generally speaking, the size of the pores in the scaffold will range from about one to ten times the diameter of the cells to be seeded therein.

Suitable living cells for use with the scaffold include, but are not limited to, epithelial cells (e.g., keratinocytes, adipocytes, hepatocytes), neurons, glial cells, astrocytes, podocytes, mammary epithelial cells, islet cells, endothelial cells (e.g., aortic, capillary and vein endothelial cells), and mesenchymal cells (e.g., dermal fibroblasts, mesothelial cells, osteoblasts), smooth muscle cells, striated muscle cells, ligament fibroblasts, tendon fibroblasts, chondrocytes, fibroblasts, and any of a variety of stem cells. Also suitable for use in the scaffold are genetically modified cells, immunologically masked cells, and the like.

It is further within the contemplation of the present invention to add tissue specific extracellular matrix (ECM) proteins to the cell scaffold. Appropriate ECM proteins may be added to the scaffold in order to further promote cell ingrowth, tissue development, and cell differentiation within the scaffold. Alternatively, the scaffold of the present invention can include ECM macromolecules in particulate form or include extracellular matrix molecules deposited by viable cells.

Extracellular matrix molecules are commercially available. For example, extracellular matrix from EHS mouse sarcoma tumor is available. (Matrigel®, Becton Dickinson, Corp. Medford, Mass.).

The term “extracellular matrix proteins” is art recognized and is intended to include one or more of fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix molecules are described in Kleinman et al., J. Biometer. Sci. Polymer Edn., 5: 1-11, (1993), herein incorporated by reference. It is intended that the term encompass presently unknown extracellular matrix proteins that may be discovered in the future, since their characterization as an extracellular matrix protein will be readily determinable by persons skilled in the art.

Additional biologically active macromolecules helpful for cell growth, morphogenesis, differentiation, and tissue building, include growth factors, proteoglycans, glycosaminoglycans and polysaccharides. These compounds are believed to contain biological, physiological, and structural information for development or regeneration of tissue structure and function. These compounds are described in the literature and are also commercially available.

For example, growth factors can be isolated from tissue using methods known to those of skill in the art. For example, growth factors can be isolated from tissue, produced by recombinant means in bacteria, yeast or mammalian cells. EGF can be isolated from the submaxillary glands of mice. Genetech (San Francisco, Calif.) produces TGF-β recombinantly. Many growth factors are also available commercially from vendors, such as Sigma Chemical Co., St. Louis, Mo.; Collaborative Research, Los Altos, Calif.; Genzyme, Cambridge, Mass.; Boehringer, Germany; R&D Systems, Minneapolis, Minn.; and GIBCO, Grand Island, N.Y. The commercially available growth factors may be obtained in both natural and recombinant forms.

The term “growth factors” is art recognized and is intended to include, but is not limited to, one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-P1, TGF β1.2, TGF-β2, TGF-β3, TGF-β5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. Additional growth factors are described in Sporn and Roberts, Peptide Growth Factors and Their Receptors I, Springer-Verlag, New York (1990) which is hereby incorporated by reference. It is intended for the term “growth factors” to encompass presently unknown growth factors that may be discovered in the future, since their characterization as a growth factor will be readily determinable by persons skilled in the art.

The term “proteoglycan” is art recognized and is intended to include one or more of decorin and dermatan sulfate proteoglycans, keratin or keratan sulfate proteoglycans, aggrecan or chondroitin sulfate proteoglycans, heparan sulfate proteoglycans, biglycan, syndecan, perlecan, or serglycin. The term “proteoglycans” encompasses presently unknown proteoglycans that may be discovered in the future, since their characterization as a proteoglycan will be readily determinable by persons skilled in the art.

The term “glycosaminoglycan” is art recognized and is intended to include one or more of heparan sulfate, chondroitin sulfate, dermatan sulfate, keratan sulfate, hyaluronic acid. The term encompasses presently unknown glycosaminoglycans that may be discovered in the future, since their characterization as a glycosaminoglycan will be readily determinable by persons skilled in the art.

The term “polysaccharide” is art recognized and is intended to include one or more of heparin, dextran sulfate, chitin, alginic acid, pectin, and xylan. The term encompasses presently unknown polysaccharides that may be discovered in the future, since their characterization as a polysaccharide will be readily determinable by persons skilled in the art.

Other biologically active agents such as nutrients, cytokines, hormones, growth factors, angiogenic factors, immunomodulatory factors, and drugs are also expected to aid the cells in thriving in the scaffold matrix. As a result, it is therefore within the scope of the present invention to include one or more of these useful compounds within the scaffold to further promote cell ingrowth and tissue development and organization within the scaffold. These are described in the literature and are also commercially available.

Furthermore, biologically active short peptide sequences derived from proteins may also be used. For example, cell adhesion may be enhanced by a number of short peptide sequences derived from adhesion proteins. These sequences are able to bind to cell-surface receptors and mediate cell adhesion with an affinity similar to that obtained with intact proteins. (Arg-Gly-Asp) (RGD) is one such peptide which may be coated onto the surfaces of three dimensional scaffolds to increase cell adhesion. This sequence binds to integrin receptors on a wide variety of cell types.

Additionally, the scaffold may be used in combination with other prostheses. For example, when used to replace or repair tubular organs, such as those in the vascular system, urogenital tract, esophagus, and bile duct, it is helpful to use a stent. A stent is a generally longitudinal tubular device which is useful to open and support various lumens in the body. These devices are implanted within the vessel to open and/or reinforce collapsing or partially occluded sections of the vessel. In one embodiment, the scaffold may partially or fully coat or circumscribe the stent.

In a still further aspect of the invention, the scaffold of the present invention may be coated with a suitable material to promote adhesion of the scaffold when implanted into a recipient. Particularly preferred is a thin layer of hyaluronic acid. The layer may be applied by any known thin coating method to all or part of an exterior surface of the scaffold. One method for coating materials with hyaluronic acid is disclosed in U.S. Pat. No. 6,129,956, the entirety of which is hereby incorporated by reference.

In one embodiment of the present invention, a scaffold for providing a lining of at least a part of the esophagus is provided. The normal esophagus has an internal mucosa layer, a sub-mucosa layer, and an external muscularis layer. In normal esophageal function, the esophageal sphincter closes after swallowing to prevent acids from the stomach from entering the esophagus. In certain medical conditions known as Gastro Esophageal Reflux Disease (GERD), the esophageal sphincter does not function properly and acids from the stomach erode the internal mucosa and sub-mucosa layers of the esophagus. When this occurs the patient has a greater than normal risk of contracting esophageal cancer, especially when the damaged tissue begins to grow pre-displastic rather than normal epithelial cells in these layers. Treatment of this condition generally involves methods which ablate the undesirable cells down to the muscularis layer and allow regrowth of normal epithelial cells. During the regrowth phase any pre-displastic cells remaining compete with normal epithelial cells to replace the tissue that has been removed. In about 10-20% of the patients receiving this treatment, abnormal cells return. The scaffold of the present invention is intended to provide a shorter more comfortable recovery period and to provide a competitive advantage to the normal epithelial cells which are seeded onto the esophageal scaffold prior to implantation.

The esophageal scaffold is formed from a polymer including alginate into a tube having an external diameter of about 16-23 mm and a thickness of from about 0.5 mm to about 2 mm. The length is dictated by the individual patient's esophagus and the area in need of repair. Desirably, the tube has an internal architecture of a gradient of pore sizes ranging from about 2 μm to about 5 μm toward an internal diameter of the tube to from about 30 μm to about 60 μm toward an external diameter of the tube. Particularly desirable is the presence of uniformly shaped pores throughout the scaffold. This design permits gas, water, and nutrients to gain access to the scaffold, allows growth of epithelial cells, but prevents loss of seeded epithelial cells, which are approximately 20 μm in diameter, from leaving the scaffold via the esophageal channel.

At least normal esophageal epithelial cells are seeded onto the cell scaffold and grown therein. Stem cells may also be used, particularly toward the exterior of the tube. Preferably, the normal cells have been previously harvested from the recipient for later use in the scaffold. The displastic cells of the diseased esophagus are removed using methods such as argon plasma coagulation (APC). The scaffold is then seeded with the epithelial cells. The scaffold may be treated with ECM proteins, growth factors, antibiotics, and the like either before, during, or after the cells are seeded. An expandable stent, such as the commercially available metallic Wallstent™ (Boston Scientific Corp., Boston, Mass.) or a self-expanding flexible knitted nitinol Strecker™ (Boston Scientific Corp., Boston, Mass.) is placed in its collapsed form into the interior or lumen of the scaffold tube. The seeded scaffold and stent assembly are then implanted into the esophagus, and the stent expanded to hold the scaffold in place against the interior wall of the esophagus. Desirably, the stent is a balloon expandable device that may be implanted with a balloon catheter and is flexible enough to conform to normal peristaltic activity. Over time, typically about 7 days, the scaffold will profuse with normal esophageal cells. The stent may then be removed and normal esophageal function will be restored. The scaffold will eventually degrade leaving behind a normal esophagus.

In a further embodiment of the present invention, a scaffold tube is used to treat GERD by first treating the scaffold with a predetermined concentration of a cell destroying compound, such as lye or a peroxide. The scaffold is dimensioned and implanted as described above, except in this instance the scaffold is used to remove the pre-displastic tissue. The scaffold will deliver the necessary amount of treating chemical while being too dilute to destroy the muscularis layer. Desirably, the scaffold will biodegrade after the dangerous cells have been destroyed. Afterwards, the esophageal scaffold as described above may be implanted to regenerate healthy esophageal tissue.

In a further embodiment of the present invention, a skin graft is formed to replace a damaged or destroyed skin layer by first forming a polymer of alginate into a sheet approximately 5 mm thick, having a first pore size on a top of the sheet and a second pore size on a bottom of the sheet. The pore sizes are formed along a gradual gradient along the thickness or cross section of the sheet. The first pore size corresponds to at least a diameter of a keratinocyte cell and the second pore size corresponds to at least a diameter of a fibroblast cell. Keratinocyte cells are seeded onto the top side of the sheet and fibroblast cells are seeded onto the bottom side of the sheet. Preferably, the seeded cells are normal cells that have been previously harvested from the recipient for use in the scaffold. The scaffold may include suitable additives such as nutrients, growth factors, and the like. The seeded sheet is then placed onto the area requiring the graft with the top side facing outwardly and the bottom side facing inwardly and touching the surface to be covered. Over time, the fibroblasts will regenerate the dermis layer of the skin while the keratinocytes will regenerate the epidermis. Ideally, the alginate scaffold will be reabsorbed and a functional layer of skin will cover the area.

In a still further embodiment of the invention, a blood vessel prosthesis is made by first forming a non-woven polymer of alginate into a tube having a predetermined internal diameter, a predetermined external diameter, and a predetermined length. The dimensions are dictated by the size of the vessel to be replaced. The internal diameter has a pore size corresponding roughly to that of an endothelial cell and the external diameter has a pore size corresponding roughly to that of a smooth muscle cell. The pore sizes are formed along an abrupt gradient along the cross section of the tube. Endothelial cells are seeded onto the interior of the tube and smooth muscle cells are seeded onto the exterior of the tube. Preferably, the normal cells have been previously harvested from the recipient and grown for use in the scaffold. An antithrombotic drug such as an anticoagulant may be added to the endothelial side of the prosthesis. Active growth factors may also be added.

Optionally, a flexible stent is inserted in its collapsed form into the lumen of the scaffold tube as described above. The seeded scaffold, or seeded scaffold and stent assembly, are then implanted into the appropriate blood vessel. If present, the stent is expanded to hold the scaffold in place against the interior wall of the vessel. Over time, the scaffold will be populated with normal endothelial cells on its interior and normal smooth muscle cells on the exterior to form functional intima and adventia layers, respectively. Desirably, the scaffold will be biodegradable and will dissolve over time leaving a normal functioning blood vessel behind.

In a further aspect of the invention, a tissue modeling kit is provided, including a cell scaffold according to the invention and a plurality of viable cells from a tissue to be modeled. The viable cells are cultured in the cell scaffold. The tissue modeling kit may be used in vitro, for example, as a model system for research. For example, the tissue modeling kit can serve as a tissue mimetic for various applications.

Alternatively, the tissue modeling kit may be used in vitro to study disease states by forming a tissue mimetic as described above, except the cells can be abnormal disease state cells such as cirrhotic liver cells or cancer cells. The cellular activity of abnormal versus normal cells can then be compared.

In addition, the tissue modeling kit can serve to prescreen test substances as potential drug candidates or the like for evaluation of specific cellular response. For example, the tissue modeling kit may be used as a diagnostic test model for determining chemotherapeutic strategies. A tissue mimetic is formed as above, except the cells are cancer cells. The mimetic is dosed with test agents and their effectiveness is determined based on their ability to kill the cancer cells. Promising agents can then be pre-screened for tissue specific toxicity by performing an in vitro toxicity test described below.

The present invention also provides a method of testing toxicity to a tissue in vitro including forming a cell scaffold according to the invention, wherein the shape of the scaffold resembles at least a portion of a tissue to be tested, culturing cells derived from the tissue in the cell scaffold, administering a predetermined dosage of a test agent to the cell scaffold, and measuring a cellular response to the dosage. Tissue specific cells for the tissue of interest are seeded onto the scaffold to form a viable tissue culture to serve as a tissue mimetic. A certain concentration of a test substance is applied to the mimetic and cellular response is measured. The cellular response can range from cell death to altered cellular activity, i.e., excretion of proteins. In this way, it may be possible to obtain relevant information regarding tissue specific toxicity without the necessity of performing extensive toxicity testing using whole animals.

It will be apparent that the present invention has been described herein with reference to certain preferred or exemplary embodiments. The preferred or exemplary embodiments described herein may be modified, changed, added to, or deviated from without departing from the intent, spirit and scope of the present invention, and it is intended that all such additions, modifications, amendments and/or deviations be included within the scope of the following claims.

We claim:

1. A three dimensional cell scaffold, comprising:
a biocompatible polymer formed from a plurality of fibers configured so as to form a non-woven three dimensional open celled matrix having a predetermined shape, a predetermined pore volume fraction, a predetermined pore size, and a predetermined pore shape, wherein said matrix includes a plurality of connections between said plurality of fibers.

2. The cell scaffold according to claim 1, wherein said biocompatible polymer is a synthetic polymer, a natural polymer, or a combination thereof.

3. The cell scaffold according to claim 2, wherein said biocompatible polymer is biodegradable, or a combination of biodegradable and biostable.

4. The cell scaffold according to claim 3, wherein said biodegradable polymer is at least one of the group consisting of poly L-lactic acid (PLA), polyglycolic acid (PGA), alginate, hyaluronic acid, and copolymers and blends thereof.

5. The cell scaffold according to claim 4, wherein said biodegradable polymer comprises alginate or collagen.

6. The cell scaffold according to claim 1, wherein said predetermined pore shape is uniform throughout said matrix.

7. The cell scaffold according to claim 6, wherein said uniform shape is substantially circular, oval or rectilinear.

8. The cell scaffold according to claim 1, wherein said predetermined pore size is in the range of from about 0.5 micron to about 100 microns.

9. The cell scaffold according to claim 8, wherein said predetermined pore size is in the range of from about 1 micron to about 50 microns.

10. The cell scaffold according to claim 1, wherein said pore volume fraction is from about 60% to about 98%

11. The cell scaffold according to claim 10, wherein said pore volume fraction is from about 80% to about 98%.

12. The cell scaffold according to claim 1, wherein said predetermined shape is selected from the group consisting of a sheet, a tube, a cylinder, a sphere, a semi-circle, a cube, a rectangle, a wedge, and an irregular shape.

13. The cell scaffold according to claim 12, wherein said predetermined shape is a tube having an interior wall, an exterior wall, and a wall thickness, said wall thickness being from about 1 micron to about 50 microns, wherein said predetermined pore size is a gradient from a first pore size to a second pore size.

14. The cell scaffold according to claim 13, wherein said first pore size is about 2 μm to about 5 μm and said second pore size is from about 30 μm to about 60 μm.

15. The cell scaffold according to claim 14, wherein said predetermined pore size comprises one of said interior wall and said exterior wall having said first pore size and the other of said interior wall and said exterior wall having said second pore size.

16. The cell scaffold according to claim 15, wherein said gradient comprises one of a substantially gradual transition from said first pore size to said second pore size across said wall thickness and a substantially abrupt transition from said first pore size to said second pore size across said wall thickness.

17. The cell scaffold according to claim 1, wherein said biocompatible polymer comprises a plurality of polymers added sequentially to form said scaffold.

18. The cell scaffold according to claim 17, wherein said plurality of polymers includes at least one biodegradable polymer A and at least one biostable polymer B.

19. The cell scaffold according to claim 18, wherein said biodegradable polymer A is selected from the group consisting of a poly L-lactic acid (PLA), a polyglycolic acid (PGA), a collagen, a zein, a casein, a gelatin, a gluten, a serum albumen, an alginate, a hyaluronic acid, and blends and copolymers thereof.

20. The cell scaffold according to claim 18, wherein said biostable polymer B is selected from the group consisting of a poly(3-hydroxyalkanoate), a poly(3-hydroxyoctanoate), a poly(3-hydroxyfatty acid), a polyphosphazene, a poly(vinyl alcohol), a polyamide, a polyester amide, a polyamino acid, a polyanhydride, a polycarbonate, a polyacrylate, a polyalkylene, a polyalkylene glycol, a polyalkylene oxide, a polyalkylene terephthalates, a polyortho ester, a polyvinyl ether, a polyvinyl ester, a polyvinyl halide, a polyester, a polylactide, a polyglyxolide, a polysiloxane, a polyurethane, a SIBS block polymers, and blends and copolymers thereof.

21. The cell scaffold according to claim 20, wherein and said biostable polymer B is a SIBS block polymer.

22. The cell scaffold according to claim 18, wherein said scaffold includes layers of polymer A (A) and polymer B (B) according to a pattern selected from the group consisting of: A-B, A-B-A, and A-B-A-B-A.

23. The cell scaffold according to claim 18, wherein said first pore size is sufficient to accommodate a diameter of an epithelial cell and said second pore size is sufficient to accommodate a diameter of a fibroblast cell.

24. The cell scaffold according to claim 12, wherein said predetermined shape is a tube and said predetermined pore size is sufficient to accommodate a diameter of an esophageal epithelial cell.

25. The cell scaffold according to claim 1, further comprising at least one biologically active agent selected from the group consisting of a nutrient, an angiogenic factor, an immunomodulatory factor, a drug, a cytokine, an extracellular protein, a proteoglycan, a glycosaminoglycan, a polysaccharide, a growth factor, and a RGD peptide.

26. The cell scaffold according to claim 25, wherein said extracellular protein is selected from the group consisting of a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, and a kalinin.

27. The cell scaffold according to claim 25, wherein said growth factor is selected from the group consisting of a platelet derived growth factor, an insulin-like growth factor, a fibroblast growth factor, a transforming growth factor, a bone morphogenic protein, a vascular endothelial growth factor, a placenta growth factor, an epidermal growth factor, an interleukin, a colony stimulating factor, a nerve growth factor, a stem cell factor, a hepatocyte growth factor, and a ciliary neurotrophic factor.

28. The cell scaffold according to claim 25, wherein said drug is at least one of the group consisting of an immunosuppressant, an anticoagulant, and an antibiotic.

29. The cell scaffold according to claim 1, further comprising a support member selected from the group consisting of a stent, a rod, a hook, a band, and a coil.

30. The cell scaffold according to claim 1, further comprising a coating.

31. The cell scaffold according to claim 30, wherein said coating comprises hyaluronic acid.

32. The cell scaffold according to claim 1, further comprising a culture material containing cells.

33. The cell scaffold according to claim 32, wherein said cells are selected from the group consisting of epithelial cells, keratinocytes, adipocytes, hepatocytes, neurons, glial cells, astrocytes, podocytes, mammary epithelial cells, islet cells, endothelial cells, mesenchymal cells, dermal fibroblasts, mesothelial cells, stem cells, osteoblasts, smooth muscle cells, striated muscle cells, ligament fibroblasts, tendon fibroblasts, and chondrocytes.

34. A method for regenerating tissue in a mammal, comprising implanting the cell scaffold of claim 1 into said mammal.

35. The method according to claim 34, wherein said biocompatible polymer is at least one of the group consisting of a poly L-lactic acid (PLA), a polyglycolic acid (PGA), an alginate, a hyaluronic acid, a cellulose, a dextran, a pullane, a chitin, a poly(3-hydroxyalkanoate), a poly(3-hydroxyoctanoate), a poly(3-hydroxyfatty acid), a collagen, a zein, a casein, a gelatin, a gluten, a serum albumen, a polyphosphazene, a polyvinyl alcohol, a polyamide, a polyester amide, a poly amino acid, a polyanhydride, a polycarbonate, a polyacrylate, a polyalkylene, a polyalkylene glycol, a polyalkylene oxide, a polyalkylene terephthalate, a polyortho ester, a polyvinyl ether, a polyvinyl ester, a polyvinyl halide, a polyester, a polylactide, a polyglyxolide, a polysiloxane, a styrene isobutyl styrene block polymer, a polyurethane, and copolymers and blends thereof.

36. The method according to claim 35, wherein said scaffold is formed from a combination of:
(a) a biodegradable polymer selected from the group consisting of a poly L-lactic acid (PLA), a polyglycolic acid (PGA), an alginate, a hyaluronic acid, and copolymers and blends thereof, and
(b) a biostable polymer selected from the group consisting of a styrene isobutyl styrene block polymer, a polyurethane, and copolymers and blends thereof.

37. The method according to claim 34, wherein said predetermined shape is selected from the group consisting of a sheet, a tube, a cylinder, a sphere, a semi-circle, a cube, a rectangle, a wedge, and an irregular shape.

38. The method according to claim 34, further comprising the step of seeding cells into said cell scaffold prior to said implanting step.

39. The method according to claim 38, wherein said cells are selected from the group consisting of epithelial cells, keratinocytes, adipocytes, hepatocytes, neurons, glial cells, astrocytes, podocytes, mammary epithelial cells, islet cells, endothelial cells, mesenchymal cells, dermal fibroblasts, mesothelial cells, stem cells, osteoblasts, smooth muscle cells, striated muscle cells, ligament fibroblasts, tendon fibroblasts, chondrocytes, and fibroblasts.

40. The method according to claim 34, wherein said tissue is selected from the group consisting of nerve, skin, vascular, cardiac, pericardial, muscle, ocular, periodontal, bone, cartilage, tendon, ligament, breast, pancreatic, esophageal, stomach, kidney, hepatic, mammary, adrenal, urological, and intestinal.

41. The method according to claim 34, further comprising the step of treating said cell scaffold with at least one biologically active agent prior to said implanting step.

42. The method according to claim 41, wherein said biologically active agent is at least one of the group consisting of an extracellular protein, a growth factor, a nutrient, an angiogenic factor, an immunomodulatory factor, a drug, a cytokine, an extracellular protein, a proteoglycan, a glycosaminoglycan, and a polysaccharide.

43. A method of treating Gastro Esophageal Reflux Disease (GERD), comprising the steps of:
forming a biocompatible polymeric matrix formed from a plurality of fibers configured so as to form a non-woven three dimensional open celled tubular matrix, said matrix having a predetermined pore volume fraction, a predetermined pore shape, and a predetermined pore size sufficient to accommodate a diameter of esophageal epithelial cells, wherein said matrix includes a plurality of connections between said plurality of fibers;
seeding said matrix with esophageal epithelial cells or stem cells; and
implanting said matrix into a mammalian esophageal space.

44. The method according to claim 43, wherein said predetermined pore size includes a gradient of pore sizes ranging from about 2 μm to about 5 μm toward an internal diameter of said tubular matrix to from about 30 μm to about 60 μm toward an external diameter of the tubular matrix.

45. The method according to claim 43, wherein said biocompatible tubular matrix is made from alginate.

46. The method according to claim 43, further comprising the step of combining a tubular stent with said biocompatible matrix prior to said implanting step.

47. The method according to claim 43, further comprising the step of coating said biocompatible matrix with hyaluronic acid prior to said implanting step.

48. The method according to claim 43, further comprising the step of administering a bioactive agent to said biocompatible matrix prior to said implanting step.

49. The method according to claim 43, wherein said cells are selected from the group consisting of autologous, xenogeneic, allogenic, and syngeneic.

50. The method according to claim 49, wherein said cells are autologous.

51. A method of removing diseased esophageal tissue, comprising the steps of:
forming a biocompatible polymeric matrix formed from a plurality of fibers configured so as to form a non-woven three dimensional tubular matrix, said matrix having a predetermined pore volume fraction, a predetermined pore shape, a predetermined pore shape and a predetermined pore size, wherein said matrix includes a plurality of connections between said plurality of fibers;
treating said matrix with a predetermined concentration of a cell destroying compound; and
implanting said matrix into a mammalian esophageal space.

52. The method according to claim 50, wherein said cell destroying compound is selected from the group consisting of a lye and a peroxide.

53. A three dimensional cell scaffold according to claim 1, formed from the steps of:
admixing at least a biocomparible polymer with a compatible solvent to form a flowable polymer mixture;
applying at least one fiber formed from said polymer mixture to a table capable of motion in at least a first plane (x) and a second plane (y) perpendicular to said first plane; and
controlling movement of at least said table so as to form said matrix.

54. A tissue modeling kit, comprising:
a cell scaffold according to claim 1; and
a plurality of viable cells from a tissue to be modeled, wherein said viable cells are cultured in said cell scaffold.

55. The tissue modeling kit according to claim 54, further comprising at least one biologically active agent selected from the group consisting of a nutrient, an angiogenic factor, an immunomodulatory factor, a drug, a cytokine, an extracellular protein, a proteoglycan, a glycosaminoglycan, a polysaccharide, a growth factor, and a RGD peptide.

56. A method of testing toxicity to a tissue, comprising:
forming a cell scaffold according to claim 1, wherein said shape resembles at least a portion of a tissue to be tested;
culturing cells derived from said tissue in said cell scaffold;
administering a predetermined dosage of a test agent to said cell scaffold; and
measuring a cellular response to said dosage.

57. The method according to claim 56, further comprising the steps of:
culturing cells derived from said tissue in a control cell scaffold;
administering a dosage of a control agent to said control scaffold;
measuring a control response to said dosage; and
comparing said cellular response to said control response.

58. The method according to claim 56, wherein said dosage is a series of dilutions of said agent, and further comprising the step of generating a dose response curve from cellular response results obtained in said measuring steps.

59. The method according to claim 56, wherein said cellular response is cell death.

60. The method according to claim 56, wherein said dosage is of a carcinogen.

61. The method according to claim 56, wherein said cells are disease state cells and said test agent is a drug candidate.

62. The method according to claim 56, wherein said cells are cancer cells.
